SEQUENCE LISTING (92 sequences)

SEQ ID NO: 1 - DNA, 19 bp, Artificial Sequence
  Synthetic nucleic acid
tcctacgggaggcagcagt

SEQ ID NO: 2 - DNA, 26 bp, Artificial Sequence
  Synthetic nucleic acid
cagggtatctaatcctgtttgctacc

SEQ ID NO: 3 - DNA, 20 bp, Artificial Sequence
  Synthetic nucleic acid
ttcaatgcaatactcgtatc

SEQ ID NO: 4 - DNA, 20 bp, Artificial Sequence
  Synthetic nucleic acid
gtacattcaatgcaatactc

SEQ ID NO: 5 - DNA, 20 bp, Artificial Sequence
  Synthetic nucleic acid
cacgtatctcattttattcc

SEQ ID NO: 6 - DNA, 22 bp, Artificial Sequence
  Synthetic nucleic acid
aatacacgtatctcattttatt

SEQ ID NO: 7 - DNA, 20 bp, Artificial Sequence
  Synthetic nucleic acid
ctcttcttcttattcttcat

SEQ ID NO: 8 - DNA, 21 bp, Artificial Sequence
  Synthetic nucleic acid
cctcttcttcttattcttcat

SEQ ID NO: 9 - DNA, 18 bp, Artificial Sequence
  Synthetic nucleic acid
gccttcgcaataggtatt

SEQ ID NO: 10 - DNA, 18 bp, Artificial Sequence
  Synthetic nucleic acid
cgccttcgcaataggtat

SEQ ID NO: 11 - DNA, 20 bp, Artificial Sequence
  Synthetic nucleic acid
attctttcccaagaaaagga

SEQ ID NO: 12 - DNA, 20 bp, Artificial Sequence
  Synthetic nucleic acid
gtcataattctttcccaaga

SEQ ID NO: 13 - DNA, 20 bp, Artificial Sequence
  Synthetic nucleic acid
caatgggtattcttcttgat

SEQ ID NO: 14 - DNA, 20 bp, Artificial Sequence
  Synthetic nucleic acid
tagttatacagtttccaacg

SEQ ID NO: 15 - DNA, 20 bp, Artificial Sequence
  Synthetic nucleic acid
ctagttatacagtttccaac

SEQ ID NO: 16 - DNA, 20 bp, Artificial Sequence
  Synthetic nucleic acid
tccagtactctagttacaca

SEQ ID NO: 17 - DNA, 19 bp, Artificial Sequence
  Synthetic nucleic acid
ccagtactctagttacaca

SEQ ID NO: 18 - DNA, 20 bp, Artificial Sequence
  Synthetic nucleic acid
tttctttcttcccaactgaa

SEQ ID NO: 19 - DNA, 20 bp, Artificial Sequence
  Synthetic nucleic acid
atttctttcttcccaactga

SEQ ID NO: 20 - DNA, 20 bp, Artificial Sequence
  Synthetic nucleic acid
tacattccgaaaaacgtcat

SEQ ID NO: 21 - DNA, 20 bp, Artificial Sequence
  Synthetic nucleic acid
ttacattccgaaaaacgtca

SEQ ID NO: 22 - DNA, 19 bp, Artificial Sequence
  Synthetic nucleic acid
tatgcagtttccaacgcaa

SEQ ID NO: 23 - DNA, 19 bp, Artificial Sequence
  Synthetic nucleic acid
ctctagttatgcagtttcc

SEQ ID NO: 24 - DNA, 20 bp, Artificial Sequence
  Synthetic nucleic acid
aagtgcttaatgaggttaag

SEQ ID NO: 25 - DNA, 20 bp, Artificial Sequence
  Synthetic nucleic acid
tttcaagtgcttaatgaggt

SEQ ID NO: 26 - DNA, 19 bp, Artificial Sequence
  Synthetic nucleic acid
gggtaaatgcaaaaaggca

SEQ ID NO: 27 - DNA, 19 bp, Artificial Sequence
  Synthetic nucleic acid
gcaaggtagatgttgagca

SEQ ID NO: 28 - DNA, 20 bp, Artificial Sequence
  Synthetic nucleic acid
ctttattcccacataaaagc

SEQ ID NO: 29 - DNA, 20 bp, Artificial Sequence
  Synthetic nucleic acid
tccttattcatgaggtacat

SEQ ID NO: 30 - DNA, 18 bp, Artificial Sequence
  Synthetic nucleic acid
aagtaccgtcactgtgtg

SEQ ID NO: 31 - DNA, 19 bp, Artificial Sequence
  Synthetic nucleic acid
ttaagtaccgtcactgtgt

SEQ ID NO: 32 - DNA, 20 bp, Artificial Sequence
  Synthetic nucleic acid
gtcaatttggcatgctatta

SEQ ID NO: 33 - DNA, 20 bp, Artificial Sequence
  Synthetic nucleic acid
tttaacgtcaatttggcatg

SEQ ID NO: 34 - DNA, 18 bp, Artificial Sequence
  Synthetic nucleic acid
cccaagcagttctatggt

SEQ ID NO: 35 - DNA, 18 bp, Artificial Sequence
  Synthetic nucleic acid
tcccaagcagttctatgg

SEQ ID NO: 36 - DNA, 20 bp, Artificial Sequence
  Synthetic nucleic acid
tacacgtacaccttattctt

SEQ ID NO: 37 - DNA, 19 bp, Artificial Sequence
  Synthetic nucleic acid
catcaatgtacacgtacac

SEQ ID NO: 38 - DNA, 19 bp, Artificial Sequence
  Synthetic nucleic acid
cattcaagaccaacagttt

SEQ ID NO: 39 - DNA, 19 bp, Artificial Sequence
  Synthetic nucleic acid
caaccattcaagaccaaca

SEQ ID NO: 40 - DNA, 18 bp, Artificial Sequence
  Synthetic nucleic acid
gcttagttgagctaagcg

SEQ ID NO: 41 - DNA, 19 bp, Artificial Sequence
  Synthetic nucleic acid
tcaaaggcagttgcttagt

SEQ ID NO: 42 - DNA, 19 bp, Artificial Sequence
  Synthetic nucleic acid
ctagctatccagttcagaa

SEQ ID NO: 43 - DNA, 19 bp, Artificial Sequence
  Synthetic nucleic acid
ctctagctatccagttcag

SEQ ID NO: 44 - DNA, 19 bp, Artificial Sequence
  Synthetic nucleic acid
cacccgttcttctcttaca

SEQ ID NO: 45 - DNA, 18 bp, Artificial Sequence
  Synthetic nucleic acid
cacccgttcttctcttac

SEQ ID NO: 46 - DNA, 20 bp, Artificial Sequence
  Synthetic nucleic acid
cagtatgaactttccattct

SEQ ID NO: 47 - DNA, 21 bp, Artificial Sequence
  Synthetic nucleic acid
acagtatgaactttccattct

SEQ ID NO: 48 - DNA, 18 bp, Artificial Sequence
  Synthetic nucleic acid
cccctcttgcactcaagt

SEQ ID NO: 49 - DNA, 18 bp, Artificial Sequence
  Synthetic nucleic acid
tctcccctcttgcactca

SEQ ID NO: 50 - DNA, 19 bp, Artificial Sequence
  Synthetic nucleic acid
tcccctcttgcactcaagt

SEQ ID NO: 51 - DNA, 17 bp, Artificial Sequence
  Synthetic nucleic acid
aagtcagcccgtaccca

SEQ ID NO: 52 - DNA, 17 bp, Artificial Sequence
  Synthetic nucleic acid
cgcactcaagtcagccc

SEQ ID NO: 53 - DNA, 19 bp, Artificial Sequence
  Synthetic nucleic acid
ctattcgcaagaaggcctt

SEQ ID NO: 54 - DNA, 18 bp, Artificial Sequence
  Synthetic nucleic acid
tattcgcaagaaggcctt

SEQ ID NO: 55 - DNA, 17 bp, Artificial Sequence
  Synthetic nucleic acid
cacaaggagcaggcctg

SEQ ID NO: 56 - DNA, 17 bp, Artificial Sequence
  Synthetic nucleic acid
ccacccacaaggagcag

SEQ ID NO: 57 - DNA, 18 bp, Artificial Sequence
  Synthetic nucleic acid
ttcgcattaggcacgttc

SEQ ID NO: 58 - DNA, 19 bp, Artificial Sequence
  Synthetic nucleic acid
ctattcgcattaggcacgt

SEQ ID NO: 59 - DNA, 18 bp, Artificial Sequence
  Synthetic nucleic acid
cacacgttcttgacttac

SEQ ID NO: 60 - DNA, 23 bp, Artificial Sequence
  Synthetic nucleic acid
cgtattaccgcggctgctggcac

SEQ ID NO: 61 - DNA, 30 bp, Artificial Sequence
  Synthetic nucleic acid
cgtgcattgtcgtgtaggttcgaccctaat

SEQ ID NO: 62 - DNA, 30 bp, Artificial Sequence
  Synthetic nucleic acid
gcagctacgttcatacctacgcaaggcatt

SEQ ID NO: 63 - DNA, 30 bp, Artificial Sequence
  Synthetic nucleic acid
gaggagataccgaatcggtcgacgacattt

SEQ ID NO: 64 - DNA, 30 bp, Artificial Sequence
  Synthetic nucleic acid
tgttgcgtgaaggtcgtgaacgattggcaa

SEQ ID NO: 65 - DNA, 30 bp, Artificial Sequence
  Synthetic nucleic acid
cccctactgagcaaacgttgcactaatgga

SEQ ID NO: 66 - DNA, 30 bp, Artificial Sequence
  Synthetic nucleic acid
aacaacgaccgagtgcatagtcacgtacga

SEQ ID NO: 67 - DNA, 30 bp, Artificial Sequence
  Synthetic nucleic acid
aggagccctaaggtattggcgagaaaagtc

SEQ ID NO: 68 - DNA, 30 bp, Artificial Sequence
  Synthetic nucleic acid
ctgagtatccgcatatcttccgaggttgca

SEQ ID NO: 69 - DNA, 30 bp, Artificial Sequence
  Synthetic nucleic acid
acttagctgaccgaaggaccataacgctgt

SEQ ID NO: 70 - DNA, 30 bp, Artificial Sequence
  Synthetic nucleic acid
tggaagggatccgtagtcaaccgttgactt

SEQ ID NO: 71 - DNA, 30 bp, Artificial Sequence
  Synthetic nucleic acid
cggatcgacatacgacgcctacagaatgtt

SEQ ID NO: 72 - DNA, 30 bp, Artificial Sequence
  Synthetic nucleic acid
taaacgtctaggcgagactatgagtgctcc

SEQ ID NO: 73 - DNA, 30 bp, Artificial Sequence
  Synthetic nucleic acid
cgtatggatcgatccgacgtaccacattag

SEQ ID NO: 74 - DNA, 30 bp, Artificial Sequence
  Synthetic nucleic acid
actgcgtatgatcgacacggctaatcgtag

SEQ ID NO: 75 - DNA, 30 bp, Artificial Sequence
  Synthetic nucleic acid
ctattcgaccagcgatatcactacgtaggc

SEQ ID NO: 76 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgaatggtaagggtcgtattagggtcgaacctacacgacaatgcacgtcgaagcggttgctaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 77 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtggtagccgattgaactaatgccttgcgtaggtatgaacgtagctgctagtcgaggccttgtatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 78 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgtcctcgatacatacgaaatgtcgtcgaccgattcggtatctcctctggcaccgaagacaaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 79 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgtgctacgctttacgcttgccaatcgttcacgaccttcacgcaacacttgatcgaaccgaaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 80 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgacaacagacggtacgtccattagtgcaacgtttgctcagtagggggtctaagcgtcacttatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 81 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggagcagttttcttctcgtacgtgactatgcactcggtcgttgttggaataccggtcgtaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 82 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgaacgcaatacggtgggacttttctcgccaataccttagggctcctgtgtacctaagcgaaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 83 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggatgacggataagttgcaacctcggaagatatgcggatactcagacgtgatatgcgcagatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 84 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggtgtacatcggatgacagcgttatggtccttcggtcagctaagtaagtccgttttccacatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 85 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgttcagtcaaccggagaagtcaacggttgactacggatcccttccatgtagagctctcacaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 86 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtggcgattgtcgcgttaaacattctgtaggcgtcgtatgtcgatccgggacttcgcttcataatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 87 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgtcgtgtgtcgtgagaggagcactcatagtctcgcctagacgtttatgacgagatatcacgatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 88 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtggtagcgtgtaacgcactaatgtggtacgtcggatcgatccatacgcaactttgtaccgagatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 89 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgagcaaccatcttgagctacgattagccgtgtcgatcatacgcagtccgaggcattactgaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 90 - DNA, 459 bp, Artificial Sequence
  Synthetic nucleic acid
gtgagaagcctacacaaacgtaacgtcagggctaagacaaacgctaacggtacaccctagatgggagcttgtagctagatcgctaagtcctaccgacatgtaggcatactcacgaaggcaattccctgaaagcctcgtcttatcccgaacttggcatctgctgatacgtcaggttgaacgcgtacatttacctgtcatgcgtgggccttctccgaatagcctacgtagtgatatcgctggtcgaataggcggattgctcataaatgcacattggctaaggcccacggaacacgaatcacgtgagatcacttactattcgacggaactactatacgcaccgggacatgcaagtagcgtcccacaagcataaggaactctatactcgccatctacgcagctacaggggatacacgtatgagcggttacgaagtaaagccgagatagagcggtctttagaga

SEQ ID NO: 91 - DNA, 25 bp, Artificial Sequence
  Synthetic nucleic acid
gagaagcctacacaaacgtaacgtc

SEQ ID NO: 92 - DNA, 23 bp, Artificial Sequence
  Synthetic nucleic acid
ctctaaagaccgctctatctcgg

(19) United States
(12) Patent Application Publication
(10) Pub. No.: US 2018/0148769 A1
(43) Pub. Date: May 31, 2018

(54) BACTERIAL FLORA ANALYSIS METHOD AND BACTERIAL FLORA ANALYSIS DEVICE

(71) Applicant: Mitsubishi Chemical Corporation, Tokyo (JP)

(72) Inventors: Ai HARA, Tokyo (JP); Hiroyuki OSHIMA, Tokyo (JP)

(73) Assignee: Mitsubishi Chemical Corporation, Tokyo (JP)

(21) Appl. No.: 15/361,250

(22) Filed: Nov. 25, 2016

(51) Int. Cl. C12Q 1/68

(52) U.S. Cl. CPC C12Q 1/689; C12Q 2600/158; C12Q 2600/16; C12Q 1/6832

(57) ABSTRACT

The object of the present invention is to provide a bacterial flora analysis method for quantitatively evaluating bacterial flora and to provide a device used in the method. The device relating to the present invention is a device mounted with (a) a probe consisting of a nucleic acid hybridizing to 16SrRNA specific to each of 1 kind or 2 or more kinds of bacteria to be a subject of detection, and at least one of (b) a total amount indicator probe and (c) one or more kinds of an absolute amount indicator probe.

### TECHNICAL FIELD OF THE INVENTION

The present invention relates to a device for bacterial flora analysis and a method for analyzing bacterial flora using the device.

### RELATED ART

The result of identifying or quantifying bacterial species contained in a sample is useful as an indicator for evaluating an environment or a health state of an animal or a plant, and there are currently several application examples including diagnosis of an infection or the like.

Examples of an evaluation method include a method in which an individual bacterium included in a sample is actually observed using a microscope or the like, bacterial species of the bacteria is identified based on morphology or the like, and each bacterium is directly counted or the like. Depending on situation, the identification efficiency may be improved by screening the bacteria using a selection medium or the like.

There is also a method in which proteins synthesized by bacteria are detected by using an antibody specific to the proteins or a method in which metabolism products are detected by using a mass analyzer.

As a technique which has the best speed and has a universal use, there is a molecular biological nucleic acid amplification technique like PCR and LAMP and a signal amplification technique like invader method. A method of selectively amplifying and detecting nucleic acids of specific sequence included in bacteria as a detection subject by using the aforementioned methods is widely used (Patent Publications 1 and 2).

Meanwhile, regarding a technique for inclusively analyzing a group of bacteria contained in a sample, i.e., so-called bacterial flora, various studies have been made for the purpose of applying the technique in evaluation of an environment, diagnosis, or the like. Examples include amplifying non-selectively a nucleic acid based on a sequence of nucleic acids that are commonly present in bacteria as a subject for detection followed by fragment analysis according to a treatment with restriction enzymes, determining a sequence by sequencer or the like after cloning, and performing identification based on hybridization with a specific sequence within a corresponding amplified sequence. For carrying out those methods, a high throughput device like electrophoresis device and DNA chip may be used (Patent Literatures 3 and 4).

### CITATION LIST

### Patent Publication

[Patent Publication 1] JP 2004-57059 A

[Patent Publication 2] JP 2007-244349 A

[Patent Publication 3] JP 2004-290171 A

[Patent Publication 4] JP 5139620 B

### SUMMARY OF THE INVENTION

### Problems to be Solved by the Invention

Bacterial flora present in the environment, for example, enterobacteria, resident bacteria of skin, oral bacteria, bacteria contained in soil, and bacteria contained in active sludge, exhibit mutual influences among various kinds of bacteria and also exhibit an influence on the existing environment itself.

In case of analyzing the bacterial flora, it takes time if a method of using culturing is used, and there is also a possibility that only a part of bacteria are detected among the entire bacteria consisting of various types. There can be also a possibility that the bacterial flora is different before and after the culture. The method of using antigen-antibody reaction or mass analysis may not be applied if the bacteria have no specific antibody or the bacteria have no previously-defined proteins or metabolites that are specific to the bacteria, and thus they are limited in terms of the type of bacteria which can be detected.

For example, to evaluate in batch mode the entire bacteria of various types that are included in bacterial flora, it is possible that the nucleic acids contained in the bacterial flora are amplified in batch mode by an amplification means such as PCR and each bacterium is selected based on specificity of hybridization to a probe using a DNA chip or the like and qualitatively evaluated. However, even with an exhaustive array, it is difficult to have quantitative evaluation of the amount of whole bacteria, considering a possibility that unknown bacteria are present in the bacterial flora. Furthermore, since the probe hybridization efficiency is different in each case, it was difficult to have quantitative evaluation.

Accordingly, object of the present invention is to provide a bacterial flora analysis method for quantitatively evaluating bacterial flora and a device used for the method.

### Means for Solving the Problems

To solve the problems described above, the inventors of the present invention conducted intensive studies. As a result, it was found that bacterial flora can be analyzed by using a probe consisting of a nucleic acid hybridizing to 16SrRNA specific to each of bacteria and a total amount indicator probe and/or an absolute amount indicator probe. The present invention is completed accordingly.

Namely, the present invention relates to a device mounted with the following probe (a) and at least one of probe (b) and (c):
(a) a probe consisting of a nucleic acid hybridizing to 16SrRNA specific to each of 1 kind or 2 or more kinds of bacteria to be a subject of detection,
(b) a total amount indicator probe,
(c) one or more kinds of an absolute amount indicator probe.

Regarding the device of the present invention, it is preferable that the probe (b) contains a base sequence which is commonly possessed by the 1 kind or 2 or more kinds of the aforementioned bacteria as a detection subject. It is also preferable that the probe (c) contains a base sequence which captures a mixture of at least one kind of a predetermined externally added control, and wherein the mixture contains the at least one kind of a predetermined externally added control and at least one kind of a predetermined externally added control at a concentration ratio of 2ⁿ (n is an arbitrary integer) relative to each other.

Herein, examples of the bacteria to be a subject of detection include enterobacteria, resident bacteria of skin, and oral bacteria.

Examples of the enterobacteria include at least one of genus Lactobacillus, genus Streptococcus, genus Veionella, genus Bacteroides, genus Eubacterium, genus Bifidobacterium, and genus Clostridium.

Examples of the resident bacteria of skin include at least one of genus Propionibacterium and genus Staphylococcus.

Examples of the oral bacteria include at least one of genus Porphyromonas, genus Tannerella, genus Treponema, genus Campylobacter, genus Fusobacterium, genus Parvimonas, genus Streptococcus, genus Aggregatibacter, genus Capnocytophaga, genus Eikenella, genus Actinomyces, genus Veillonella, genus Selenomonas, genus Lactobacillus, genus Pseudomonas, genus Haemophilus, genus Klebsiella, genus Serratia, genus Moraxella, and genus Candida.

Furthermore, more specific examples of the oral bacteria include at least one of Porphyromonas gingivalis, Tannerella forsythia, Treponema denticola, Campylobacter gracilis, Campylobacter rectus, Campylobacter showae, Fusobacterium nucleatum subsp. vincentii, Fusobacterium nucleatum subsp. polymorphum, Fusobacterium nucleatum subsp. animalis, Fusobacterium nucleatum subsp. nucleatum, Fusobacterium periodonticum, Parvimonas micra, Prevotella intermedia, Prevotella nigrescens, Streptococcus constellatus, Aggregatibacter actinomycetemcomitans, Campylobacter concisus, Capnocytophaga gingivalis, Capnocytophaga ochracea, Capnocytophaga sputigena, Eikenella corrodens, Streptococcus gordonii, Streptococcus intermedius, Streptococcus mitis, Streptococcus mitis by 2, Actinomyces odontolyticus, Veillonella parvula, Actinomyces naeslundii II, Selenomonas noxia, and Selenomonas noxia.

Furthermore, regarding the device of the present invention, it is preferable that the probe (a) is any one sequence of the following sequences:
(i) at least 1 sequences selected from base sequences represented by SEQ ID NOs: 3 to 59
(ii) a sequence complementary to (i)
(iii) a sequence substantially the same as the sequence of (i) or (ii).

Furthermore, the device of the present invention can be a fiber type micro array.

In addition, the present invention also relates to a probe set for detecting oral bacteria, which comprises any one sequence of the following sequences:
(i) at least 1 sequences selected from base sequences represented by SEQ ID NOs: 3 to 59, and a base sequences represented by SEQ ID NO: 60
(ii) a sequence complementary to (i)
(iii) a sequence which comprises the sequence (i) or (ii) in the part
(iv) a sequence as a part of the sequence (i) or (ii)

The present invention also relates to a method for estimating an absolute amount of bacterial species as a subject of detection including the following steps:
(1) a step of obtaining, for previously isolated each bacteria as a subject of detection, a coefficient from signal intensity ratio of a probe for detecting the bacteria as a subject of detection,
(2) a step of calculating copy number of each bacterial species as a subject of detection based on data that is obtained from a test sample using a hybridization efficiency coefficient, in which the value obtained in the step (1) is used as a hybridization efficiency coefficient, and
(3) a step of comparing signal intensity after calculation which has been calculated in the step (2) to signal intensity of an absolute amount indicator probe.

### Effects of the Invention

By using a device mounted with bacterial species-specific probe and one or both of a total amount indicator probe and an absolute amount indicator probe in a sequence of an amplification product, it becomes possible to have absolute batch mode quantification of plural kinds of bacteria present in bacterial flora. It is also possible to have quantification of the total amount of bacteria contained in a sample.

### EMBODIMENTS OF THE INVENTION

Hereinbelow, the present invention is explained in detail. Scope of the present invention is not limited to those explanations, and regarding those other than the following exemplifications, a suitable modification can be carried out within a range in which the spirit of the present invention is not adversely affected. Further, all of the publications, for example, the prior art documents, the laid-open publications, the patent publications and the other patent documents cited in the present specification are incorporated herein by reference.

The method for estimating an absolute amount (i.e., absolute amount estimation method) of bacterial species as a subject of detection according to the present invention includes the following steps:
(1) a step of obtaining, for previously isolated each bacteria as a subject of detection, a coefficient from signal intensity ratio of a probe for detecting the bacteria as a subject of detection,
(2) a step of calculating copy number of each bacterial species as a subject of detection based on data that is obtained from a test sample using a hybridization efficiency coefficient, in which the value obtained in the step (1) is used as a hybridization efficiency coefficient, and
(3) a step of comparing signal intensity after calculation which has been calculated in the step (2) to signal intensity of an absolute amount indicator probe.

Herein, the device which can be used for the method for estimating an absolute amount according to present invention is a device that is mounted with the following probe (a) and at least one of probe (b) and (c):
(a) a probe consisting of a nucleic acid hybridizing to 16SrRNA specific to each of plural kinds of bacteria to be a subject of detection,
(b) a total amount indicator probe,
(c) one or more kinds of an absolute amount indicator probe.

### Device

Any kind of device can be used as long as the device is of an array type in which a probe is fixed independently and also to enable specification of position.

The array type is not particularly limited, either, but for the purpose of stably obtaining a signal from a total amount indicator probe described below, it is better to have an array with a large amount of fixed probes instead of simple fixing of probes on a flat substrate. As for the array, a DNA micro array is preferable. In particular, a fiber type DNA micro array for fixing a probe three-dimensionally via a gel can be mentioned. Regarding the device, the form of the support is not particularly limited, and any form such as a plate, a rod and a bead may be used. In the case in which a plate is used as the support, predetermined probes can be fixed every kind on the plate with a predetermined interval (the spotting method and the like; see Science 270, 467-470 (1995) and the like). In addition, predetermined probes can be sequentially synthesized according to kind at certain positions on the plate (the photolithography method and the like; see Science 251, 767-773 (1991) and the like).

Examples of the other preferable forms of the support include those using hollow fibers. In the case in which hollow fibers are used as the support, preferably exemplified may be a device obtained by fixing every kind of the oligonucleotide probes onto each hollow fiber, and bundling and fixing all of the hollow fibers, and then repeating cutting them in the vertical direction against the longitudinal direction of the fiber. The device can be explained as a type in which the oligonucleotide probes are fixed on a through-hole substrate (see Japanese Patent No. 3510882 and the like, FIG. 1).

A method for fixing the probes on the support is not particularly limited, and the probes may be fixed on the support in any fixing mode. In addition, the method is not limited to direct fixing on the support, but, for example, the support may be previously coating-treated with a polymer such as polylysine, and the probe may be fixed on the support after the treatment. Further, in the case in which a tubular body such as a hollow fiber is used as the support, a gelatinous substance may be kept on the tubular body, and the probes may be fixed on the gelatinous substance.

### Probe

In the present specification, capturing a nucleic acid derived from bacteria based on a hybridization reaction, measuring the captured nucleic acid based on fluorescence, chemical luminescence, coloration, RI or the like, and determining the presence or absence of bacteria or measuring the amount of bacteria are referred to as detection.

A probe is to capture a nucleic acid included in bacteria as a detection subject by hybridization, and an oligo DNA consisting of complementary sequence of a sequence which is specific to the nucleic acid as a capturing subject is generally used. However, as long as it can hybridize with a sequence which is specific to the nucleic acid as a capturing subject, it may be either DNA or RNA. It may also contain PNA or LNA. cDNA may be immobilized depending on use.

When a device mounted in batch mode with plural kinds of probe is used (for example, DNA micro array), plural kinds of probe are provided to a hybridization reaction at same conditions. Thus, it is important to have constant hybridization efficiency among the probes. For such purpose, it is necessary to set Tm at constant value within a possible range by adjusting GC content or sequence length at the time of designing probes. Type or number of the probes to be mounted in a device is not particularly limited.

Bacteria specific probe (i.e., the probe (a)) contains, in a base sequence to be a template or a base sequence of a probe, a specific base sequence depending on type of bacteria.

For hybridizing a sample on a device (e.g., DNA micro array), a nucleic acid amplification method like PCR and LAMP method is applied in many cases to improve detection sensitivity and to add a detectable label. For an amplification reaction, it is important to amplify a region required for hybridization to a device during following steps and, from the viewpoint of the cost related to reagents or the like, a region which is not required for hybridization is preferably not amplified as much as possible.

According to the aforementioned PCR method, only a region located between a pair of primers is amplified, and thus it is suitable for carrying out the amplification after excluding the sequences that are not required for hybridization. As described herein, the term “pair” means a set of primers that are minimally required for an amplification reaction, and type of the pair may be different depending on a method for amplification reaction. For general PCR, the pair of primers consist of two kinds of oligo DNA, i.e., forward primer and reverse primer.

A specific primer means that a sequence to be an amplification subject is limited, and a pair of primers is not necessarily a single pair. If necessary, it is also possible to apply a multiplex method in which 2 or more pairs of primer pair are used. For example, it is possible to have a pair of primers for bacterial amplification (SEQ ID NO: 1, 2) or a pair of primers for externally added control (SEQ ID NO: 91, 92).

When bacterial flora analysis is performed by using the above device (e.g., DNA micro array), the use is not particularly limited. For a use like examination or diagnosis of a health state, enterobacterial flora, oral bacterial flora, resident bacteria of skin, or the like may be considered. Furthermore, for a use like examination of an environment, the device can be used for bacterial flora in soil, active sludge resulting from water treatment, or the like.

Among them, for a case of performing an evaluation of enterobacterial flora, bacteria of genus Lactobacillus, genus Streptococcus, genus Veionella, genus Bacteroides, genus Eubacterium, genus Bifidobacterium, or genus Clostridium may be used as bacterial species for subject detection.

For a case of performing an evaluation of resident bacteria of skin, bacteria of genus Propionibacterium (e.g., Propionibacterium acnes) or genus Staphylococcus may be used as bacterial species for subject detection.

For a case of performing an evaluation of oral bacterial flora, bacteria of genus Porphyromonas, genus Tannerella, genus Treponema, genus Campylobacter, genus Fusobacterium, genus Parvimonas, genus Streptococcus, genus Aggregatibacter, genus Capnocytophaga, genus Eikenella, genus Actinomyces, genus Veillonella, genus Selenomonas, genus Lactobacillus, genus Pseudomonas, genus Haemophilus, genus Klebsiella, genus Serratia, genus Moraxella, or genus Candida may be used as bacterial species for subject detection. More specifically, it is preferable that bacteria like Porphyromonas gingivalis, Tannerella forsythia, Treponema denticola, Prevotella intermedia, Aggregatibacter actinomycetemcomitans, Fusobacterium nucleatum subsp. vincentii, Fusobacterium nucleatum subsp. polymorphum, Fusobacterium nucleatum subsp. animalis, Fusobacterium nucleatum subsp. nucleatum, Streptococcus mutans, Streptococcus salivarius, Streptococcus sanguis, Streptococcus miris, Actinomyces viscosus, Lactobacillus gasseri, Lactobacillus phage, Lactobacillus casei, Lactobacillus delbrueckii subsp. bulgaricus, Streptococcus aureus, Pseudomonas aeruginosa, Streptococcus pyogenes, Streptococcus pneumoniae, Haemophilus influenzae, Klebsiella pneumoniae, Serratia marcescens, Serratia macescens, Moraxella catarrhalis, Candida albicans, Campylobacter gracilis, Campylobacter rectus, Campylobacter showae, Fusobacterium periodonticum, Parvimonas micra, Prevotella nigrescens, Streptococcus constellatus, Campylobacter concisus, Capnocytophaga gingivalis, Capnocytophaga ochracea, Capnocytophaga sputigena, Eikenella corrodens, Streptococcus gordonii, Streptococcus intermedius, Streptococcus mitis by 2, Actinomyces odontolyticus, Veillonella parvula, Actinomyces naeslundii II, and Selenomonas noxia, which are currently considered to be involved with periodontal disease, cavity, or opportunistic infection, may be used as bacterial species for subject detection. More preferred are Porphyromonas gingivalis, Tannerella forsythia, and Treponema denticola. Particularly preferred is Porphyromonas gingivalis.

With regard to those described above, when an amplification is carried out by using the primer shown in Table 1 below (SEQ ID NO: 1, 2), it is possible to use the sequence described in SEQ ID NOs: 3 to 59 as a probe. It is preferable to use at least one sequences that are selected from base sequences represented by SEQ ID NOs: 3 to 59. It may be a complementary sequence of at least one sequences that are selected from base sequences represented by SEQ ID NOs: 3 to 59, a sequence which is substantially the same as at least one sequences that are selected from base sequences represented by SEQ ID NOs: 3 to 59, or a sequence which is substantially the same as the complementary sequence of at least one sequences that are selected from base sequences represented by SEQ ID NOs: 3 to 59.

Herein, the “substantially the same” indicates the length which is sufficient for having specific hybridization to the sequence described in SEQ ID NOs: 3 to 59 or a complementary sequence thereof under stringent conditions.

The length of a probe is a sequence with 15 bases or more, in general. Preferably, it is a sequence with 17 bases or more, and more preferably 20 bases or more. Herein, the “stringent conditions” indicate conditions at which a specific hybridization is formed but a non-specific hybridization is not formed. Namely, it indicates conditions at which a pair of polynucleotides having high homology (homology or identity of 95% or more, preferably 96% or more, more preferably 97% or more, even more preferably 98% or more, and most preferably 99% or more) can hybridize. More specifically, such conditions can be set at the conditions that are employed for a Northern blotting method, a dot blotting method, a colony hybridization method, a plaque hybridization method, or a Southern hybridization method. Specifically, the stringent conditions can be achieved by performing hybridization at 65° C. in the presence of 0.7 to 1 M NaCl by using a membrane immobilized with polynucleotides and washing the membrane at 65° C. by using a SSC (Saline Sodium Citrate; 150 mM sodium chloride, 15 mM sodium citrate) solution with 0.1 to 2 times concentration.

At the above conditions, it is expected to obtain efficiently polynucleotides having high homology to the extent that even the temperature can be increased. Meanwhile, several factors like temperature and salt concentration may be considered as a factor for influencing the stringency of hybridization, and it is possible for a skilled person in the art to achieve constant stringency by suitably selecting those factors.

Total amount indicator probe (the above probe (b))

Even when there are many types of a probe to be mounted in a device (for example, DNA micro array), there is a high possibility of having a mixture of unidentified bacteria present in a test sample, and in nucleic acids to be amplified, nucleic acids derived from bacteria that are not a detection subject for which no specific probe is mounted (referred to as “bacteria as a non-subject for detection”) are present.

As such, it is difficult to detect every kind of bacteria as a subject for detection. For analyzing bacterial flora, it is also very important to determine the total amount of bacteria considering the ratio of the bacteria as a subject for detection in the entire bacterial flora including bacteria as a non-subject for detection and also considering the amount of the bacteria as a subject for detection that are originally found in a sample.

The bacteria as a non-subject for detection can be understood as a sum of (i) bacteria with known identity, but not required to be taken as a subject for detection and (ii) bacteria of which presence or type is unknown. Meanwhile, with regard to the (ii), as the presence is unknown and specific sequence is unclear, it cannot be clearly said that they can be certainly detected in a real case. Thus, among the bacteria of (ii), bacteria that can be amplified belong to the bacteria as a subject for detection including bacteria as a non-subject for detection. Because the bacteria that can be amplified always have a sequence complementary to a primer sequence, it can be hybridized to a probe which has the same sequence as such primer.

Thus, the all bacteria as a subject for detection described in the present invention means bacteria that can be amplified with a specific pair of primers.

For evaluating the total amount of bacteria as described above, it is also possible to measure the number of bacteria, separately from a device (DNA micro array). In that case, from the viewpoint of enhancing the convenience of operation, it is advantageous to have a probe as an indicator of total amount of bacteria mounted in a device. With regard to the probe, among the base sequences that are amplified by a pair of primers, a base sequence common to various kinds of bacterial species may be used. When such sequence is not found, it is possible that several relatively common sequences are designed and, by determining them collectively, they can be used as a total amount indicator probe. The total amount indicator probe is preferably a probe which can hybridize with a nucleic acid derived from bacteria contained in a test sample, and specifically a probe which contains, among the base sequences amplified by the above specific pair of primers, a base sequence that is commonly possessed by plural kinds of bacteria as a subject for detection.

The total amount indicator represents a total amount of amplified product which is specific to an individual bacterial species. Accordingly, as it generally has a large amount, the signal intensity may easily have a limit point (i.e., signal higher than the acceptable range of detectable signal intensity is shown).

To prevent such situations, it is possible to reduce the hybridization efficiency compared to a probe for capturing an amplification product which is specific to individual bacterial species. For carrying out PCR, 2 kinds of oligo DNA are used as one pair of primers in which one of them is fluorescent-labeled and the other is used basically with no labeling. For example, when DNA identical to the unlabeled oligo DNA is used as a probe, all of the amplified PCR products will be hybridized so that this hybridization signal is measured. For designing a probe, Tin value of the probe is lowered, for example. Specifically, it may be considered to have a method for reducing the GC content or shortening the sequence length of a probe itself.

Furthermore, during the hybridization, by adding a nucleic acid which can competitively acts against the hybridization between the amplified nucleic acid and total amount indicator probe, the signal intensity may be decreased. Examples of such nucleic acid include a nucleic acid which has the (partially) same sequence as the total amount indicator probe and a nucleic acid which has (partially) a complementary sequence of the total amount indicator probe.

Meanwhile, it is also possible to use a sequence which is the same or partially the same as the pair of primers as a total amount indicator probe. For example, in a case in which a fluorescent label is present on a forward primer, by using a probe which contains the whole or part of the sequence constituting the reverse primer, the total amount of the amplified product can be evaluated.

In a case in which a fluorescent label is present on a forward primer, by subjecting a chain which is elongated from the forward primer to the hybridization, signal detection is made. In such case, as there is a large amount of amplification product as a subject for capturing by the probe as described above, it is worried that the signal intensity may reach the limit point. However, as described above, by adjusting the probe length or GC content, the influence may be suppressed.

### Hybridization Efficiency Coefficient

With regard to the hybridization efficiency coefficient in the present specification, the hybridization efficiency coefficient for each probe is obtained from the signal intensity of a probe for each bacterial species. For a probe which can have hybridization with plural bacteria, the average value is used as a hybridization efficiency coefficient.

### Externally Added Control

In the present specification, the externally added control means a nucleic acid which is artificially designed such that the DNA of bacteria included in a test sample is not amplified, and it indicates a nucleic acid which is added in a constant amount to a sample before the amplification reaction or hybridization reaction. The externally added control corresponds to a nucleic acid which is certainly amplified at the time of performing a general amplification reaction, and it functions as a so-called a positive control. Thus, when a probe specific to the externally added control is mounted in a device (for example, DNA micro array), evaluation can be made based on detection results to see whether or not the amplification reaction or hybridization reaction has been suitably carried out.

Furthermore, when several externally added controls are designed and a mixture having different concentration for each of those controls is used, by matching with a detection result for the corresponding concentration, for example, fluorescence intensity, the bacteria contained in a sample or total amount of the bacteria can be quantified.

When the externally added control is added before an amplification reaction, it is necessary that the externally added control is a nucleic acid to be amplified by the aforementioned specific primer pair, i.e., the externally added control has a base sequence which is complementary to the primer pair. Furthermore, to have detection based on hybridization, it is necessary that the externally added control has a base sequence that is not included in any one of bacteria as a subject for detection and bacteria as a non-subject for detection.

### Designing of Externally Added Control

For example, by using the RNDBETWEEN function of “EXCEL” software by MICROSOFT, an integer from 1 to 4 is randomly generated in the number of X (X is an arbitrary number), and by connecting them, a numerical value of X digits only consisting of the number from 1 to 4 is obtained, and by replacing 1 with A, 2 with T, 3 with C, and G with 4, several random sequences with X bases of ATGC can be obtained. For those sequences, by selecting only the sequences in which the sum of G and T equals to the sum of A and T, carrying out Blast search of those selected sequences against a database like GenBank of NCBI, selecting those with less similar sequences, and adding a primer sequence to both ends of the sequence, designing can be achieved. It is also possible to elongate the designed sequence according to suitable linking or to shorten the sequence according to partial elimination of the designed sequence. When bacteria as a subject for detection is quantified by using several externally added controls, to adjust the reaction efficiency at constant level as much as possible during an amplification reaction, it is preferable not to have any big difference compared to the length of a base to be amplified from bacteria as a subject for detection. For example, if an amplified product of bacteria as a subject for detection is 500 bp or so, the amplified product of an externally added control is preferably between 300 bp and 1000 bp or so. Meanwhile, when the length of amplified chain is confirmed after amplification by using electrophoresis or the like, it is also possible that, after having a design to obtain an amplification product which has a different length compared to the bacteria as a subject for detection, the amplification product derived from the externally added control is detected at a position which is different from the band of the bacteria as a subject for detection, and the success or failure of the amplification is determined before the hybridization.

Furthermore, when several externally added controls are used, it is preferable that each externally added control consists of the same base sequence other than the sequence captured by a probe or a region around such sequence. Case in which amplification reaction is carried out by PCR or the like after adding externally added control

One or more kinds of the absolute amount indicator probe (i.e., the probe (c)) is a probe which is amplified by using a specific pair of primers, and it is a probe which does not hybridize with any nucleic acid derived from bacteria contained in a test sample but hybridizes with a nucleic acid of an externally added control prepared as a control.

The absolute amount indicator probe is contained in a mixture of a predetermined externally added control 1 and at least one externally added control 2 which is different from the externally added control 1. In this case, the externally added control 2 is mixed at concentration ratio of 2ⁿ (n is an arbitrary integer) relative to the externally added control 1. That is because the PCR itself yields an amplification product in an amount of 2n times after n cycles. Although the externally added control is 2 or more types, it is preferably 50 or less types.

Furthermore, considering that the dynamic range of a device (for example, DNA micro array) is 10⁴ or so, and when the concentration step is prepared to 15 kinds with a step of 2ⁿ times, because 2¹⁵>10⁴, it can be a sufficient indicator for the dynamic range of a device (for example, DNA micro array). However, when several externally added controls are prepared for the concentration step each with 15 kinds, it is also possible to prepare more kinds of the externally added control. Thus, considering a case in which 3 kinds of the externally added control are prepared for each concentration step, it is preferable that 45 kinds or so of the externally added control are mixed at concentration steps of 2ⁿ.

In the present invention, when several kinds of the absolute amount indicator probe are used, nucleic acids of several kinds of the absolute amount indicator probe are amplified and several kinds of the externally added control are detected by each probe. For a case in which k kinds (i.e., from number 1 to number k) of the externally added control are used, the concentration ratio from the number 1 to number k−1 and the concentration ratio from the number 1 to number k can be expressed with 2ⁿ (n is an arbitrary integer).

For example, when n from the number 1 to the number 2 is 3 and n from the number 1 to the number 3 is also 3, the concentration ratio from the number 1 to number 2 and also the concentration ratio from the number 1 to number 3 are 2³=8 times so as to have the same concentration ratio. Furthermore, when n from the number 1 to the number 2 is 2 and n from the number 1 to the number 3 is 3, it is possible to have different concentration ratio.

Namely, several kinds of the absolute amount indicator probe can be used at the same concentration or different concentration.

Basically, several kinds of the absolute amount indicator probe can be added in any combination. In case of 15 kinds, 7 kinds of a probe with concentration 1 can be added and 8 kinds of a probe with concentration 2 (i.e., probe with concentration ratio of 2 compared to concentration 1) can be added or 7 kinds of a probe with concentration 1 can be added and 8 kinds of a probe with concentration 4 can be added. It is also possible that 2 kinds of a probe with concentration 1 are added, 3 kinds of a probe with concentration 4 are added, and 1 kind of a probe with concentration 8 is added As shown in the following examples, it is also possible that 1 kind of a probe with concentration 1 is added, 1 kind of a probe with concentration 2 is added, 1 kind of a probe with concentration 4 is added, 1 kind of a probe with concentration 8 is added, . . . and 1 kind of a probe with concentration 15 is added.

To establish a calibration curve, it is necessary to have 2 or more steps of concentration, and in this regard, it is sufficient that such 2 steps are present at the concentration ratio of 2ⁿ (n is an arbitrary integer).

Meanwhile, when the externally added control to be contained in a sample has excessively high concentration, competition becomes high during an amplification reaction with bacteria as a subject for detection, and thus there is a possibility of not detecting the bacteria as a subject for detection, which are originally detectable. Thus, it is necessary to suitably control the concentration depending on application.

To detect several externally added controls by hybridization, it is necessary that a complementary sequence having a sequence specific to each of several externally added controls is individually mounted, as a probe, in a device (for example, DNA micro array).

When the presence or absence or the amount of bacterial species as a subject for detection contained in bacterial flora of actual unknown sample (i.e., real sample) is analyzed by using the above device (for example, DNA micro array), it is also possible that, for each of the probe for the bacterial species as a subject for detection contained in the device, by carrying out a treatment with the coefficient for calibrating based on a signal intensity of the absolute amount indicator probe and the signal intensity of the total amount indicator probe, the absolute quantification of the nucleic acid derived from bacteria as a subject for detection can be achieved.

### Method for Estimating Absolute Amount of Bacterial Species as Subject for Detection

### Bacteria as Subject for Detection

The possibility of having unknown bacteria in bacterial flora cannot be denied. For such reasons, it is difficult to have all kinds of bacterial species as a subject for detection by using a device (for example, DNA micro array). The subject can be a bacterium suitable for detection to evaluate a certain phenomenon among the bacteria which have been already completely identified. For example, there are several hundred kinds of oral bacteria, and many of them have been already identified. However, for evaluation a periodontal disease, it cannot be said that those identified bacteria are always suitable as a subject for detection.

Thus, for evaluating a periodontal disease, bacteria present in a periodontal pocket, for example, Porphyromonas gingivalis, Tannerella forsythia, Treponema denticola, Prevotella intermedia, Aggregatibacter actinomycetemcomitans, Fusobacterium nucleatum subsp. vincentii, Fusobacterium nucleatum subsp. polymorphum, Fusobacterium nucleatum subsp. animalis, and Fusobacterium nucleatum subsp. nucleatum may be suitable bacteria for evaluating a periodontal disease. Porphyromonas gingivalis, Tannerella forsythia, and Treponema denticola are more preferable, and Porphyromonas gingivalis is particularly preferable for evaluating a periodontal disease.

For each of those bacteria, a sequence specific to the corresponding bacteria is selected as a probe among the base sequences that are amplified by the above specific primer pair. Namely, because the base sequences amplified by the specific primer pair include those usable as a probe and those not usable as a probe, only the usable probe is selected. Basically, several kinds of bacteria are amplified using 1 kind of primer pair, and by using a part within an amplified sequence from which sequence variety is observed according to bacterial species, a probe can be prepared.

A probe having the same working effect as the sequence which can be originally used as a probe can be also used as a probe with substantially the same sequence.

As such, the bacteria as a subject for detection need to have a nucleic acid sequence that can be amplified by the above specific primer pair, and to have detection of the bacteria based on those nucleic acids, it is necessary to have, in an amplified sequence, a bacterial species-specific sequence that is different from other bacteria.

Meanwhile, such specificity may be related to batch detection of the bacteria of the same genus based on the specificity at genus level. Or, it may be the specificity which can be detected at individual species level, and it can be suitably determined depending on the object of bacterial flora analysis.

When several bacteria are amplified in batch mode and detection is made based on a specific sequence, it is necessary that the primer pair is designed based on a sequence preserved among the bacterial species. It is also necessary to use a gene having bacteria-specific sequence as a template (i.e., region amplified by primer). Examples of the gene to be a template include 16SrRNA. In the present invention, 16SrRNA itself can be a subject for detection, and it is also possible that 16SrRNA of genomic DNA as a bases for transcription is used as a subject for detection.

For example, bacteria as a subject for detection are isolated first. At that time, it is also possible to use bacteria which are already commercially available as a bacterial strain. Furthermore, the bacteria may be either alive or dead. Furthermore, instead of bacteria itself, they may be a genomic DNA extracted from bacteria.

(1) A step of obtaining, for each of bacteria previously isolated as a subject of detection, a coefficient from a signal intensity ratio of a probe for detecting the bacteria as a subject of detection.

The signal intensities after hybridization of each bacterium genomic DNA are measured in some DNA contents. There was confirmed a correlation between PCR template DNA content and signal intensity after hybridization. Thus, the standard curves of each bacterium and total bacteria can be obtained.

(2) A step of correcting a signal intensity obtained from measuring a test sample by comparing signal intensity from the test sample to a signal intensity of an absolute amount indicator probe.

The correcting factor of each DNA chip is calculated based on a property that the signal intensity of an absolute amount indicator probe consistently shows the same value. When comparing the signal intensities among different DNA chips, correction is made by using a correction coefficient for the signal intensity of the subject bacteria

(3) A step of calculating copy number of each bacterial species as a subject of detection based on a corrected signal intensity of a test sample in step (2) using the coefficient obtained in step (1),

Hereinbelow, the present invention is more specifically explained in view of the examples. However, the present invention is not limited to those examples at all.

### EXAMPLE 1

### <Production of Device and Primer>

Sequence data of 16SrRNA was downloaded from NCBI, and by using 2 sequences that are highly preserved among different bacterial species as a forward primer (SEQ ID NO: 1) and a reverse primer (SEQ ID NO: 2) shown in Table 1, DNA synthesis (LifeTechnologies) was carried out. Furthermore, fluorescent labeling using Cy5 was performed for the forward primer at that time. With regard to those primers, the forward primer was prepared at concentration of 50 pmol/μL and the reverse primer was prepared at concentration of 10 pmol/μL, respectively.

TABLE 1

| SEQ | | | |
|---|---|---|---|
| ID | | | |
| NO. | Function | Remarks | Sequence (5′→3′) |
| 1 | Forward primer | Fluores- | TCCTACGGGAGGCAGCAGT |
| | (for amplifica- | cent | |
| | tion of | label | |
| | bacteria) | at 5′ | |
| 2 | Reverse primer | | CAGGGTATCTAATCCTGTT |
| | (for amplifica- | | TGCTACC |
| | tion of | | |
| | bacteria) | | |
| 91 | Forward primer | Fluores- | GAGAAGCCTACACAAACGT |
| | (for amplifica- | cent | AACGTC |
| | tion of | label | |
| | externally | at 5′ | |
| | added control) | | |
| 92 | Reverse primer | | CTCTAAAGACCGCTCTATC |
| | (for amplifica- | | TCGG |
| | tion of | | |
| | externally | | |
| | added control) | | |

Based on each genomic DNA of oral bacteria as subject for detection (Porphyromonas gingivalis (P.g.), Tannerella forsythia (T.f.), Treponema denticola (T.d.), Campylobacter gracilis (C.gr.), Campylobacter rectus (C.r.), Campylobacter showae (C.sh.), Fusobacterium nucleatum subsp. vincentii (F.n.v), Fusobacterium nucleatum subsp. polymorphum (F.n.p), Fusobacterium nucleatum subsp. animalis (F.n.a), Fusobacterium nucleatum subsp. nucleatum (F.n.a), Fusobacterium periodonticum (F.p.), Parvimonas micra (P.m.), Prevotella intermedia (P.i.), Prevotella nigrescens (P.n.), Streptococcus constellatus (S.c.), Aggregatibacter actinomycetemcomitans (A.a.), Campylobacter concisus (C.c.), Capnocytophaga gingivalis (C.gi.), Capnocytophaga ochracea (C.o.), Capnocytophaga sputigena (C.sp.), Eikenella corrodens (E.c.), Streptococcus gordonii (S.g.), Streptococcus intermedius (S.i.), Streptococcus mitis (S.m.), Streptococcus mitis by 2 (S.mb.), Actinomyces odontolyticus (A.o.), Veillonella parvula (V.p.), Actinomyces naeslundii II (A.n.), and Selenomonas noxia (S.n.)), a sequence of 20 bases was designed while the range to be amplified by the above primers is moved by 1 base.

Next, among the base sequences designed from the above, those having GC content of about 50% were extracted. All sequences of the extracted were subjected to a homology search using the BLAST program of NCBI while l6SrRNA sequence for entire bacterial species is taken as a masking database. Accordingly, 2 sequences showing the highest sequence specificity were extracted. The extracted sequences were compared among the bacterial species, and for the sequence having higher GC content than others, 1 base or 2 bases were added at the front and end of the sequence. For the sequence having lower GC content, 1 base or 2 bases were removed.

To prepare the obtained sequence as a probe, an oligo DNA having a vinyl group at 5-terminal was synthesized (SEQ ID NOs: 3 to 59). Separately, among the base sequences that are amplified by the above specific primer pair, for the base sequence which is commonly found in plural kinds of bacteria to be a subject for detection, an oligo DNA having a vinyl group at 5-terminal was synthesized (SEQ ID NO: 60).

Furthermore, 15 kinds of a probe consisting of a sequence not included in 16SrRNA were prepared (SEQ ID NOs: 61 to 75), and an oligo DNA having a vinyl group at 5-terminal was also synthesized for them.

By using all of those kinds of oligo DNA having a vinyl group at 5-terminal and platform of DNA chip Genopal manufactured by Mitsubishi Rayon Co., Ltd., an array was prepared. All of the designed probe sequences are shown in Table 2.

TABLE 2

| | | Other | |
|---|---|---|---|
| | | bacteria | |
| SEQ | | as de- | |
| ID | | tection | Sequence |
| NO. | Function | subject | (5′→3′) |
| 3 | Probe for Porphyromonas gingivalis | | TTCAATGCAATACTCGTATC |
| | (P.g.) | | |
| 4 | Probe for Porphyromonas gingivalis | | GTACATTCAATGCAATACTC |
| | (P.g.) | | |
| 5 | Probe for Tannerella forsythia (T.f.) | | CACGTATCTCATTTTATTCC |
| 6 | Probe for Tannerella forsythia (T.f.) | | AATACACGTATCTCATTTTATT |
| 7 | Probe for Treponema denticola (T.d.) | | CTCTTCTTCTTATTCTTCAT |
| 8 | Probe for Treponema denticola (T.d.) | | CCTCTTCTTCTTATTCTTCAT |
| 9 | Probe for Campylobacter gracilis (C.g.) | | GCCTTCGCAATAGGTATT |
| 10 | Probe for Campylobacter gracilis (C.g.) | | CGCCTTCGCAATAGGTAT |
| 11 | Probe for Campylobacter rectus (C.r.) | | ATTCTTTCCCAAGAAAAGGA |
| 12 | Probe for Campylobacter rectus (C.r.) | C.s. | GTCATAATTCTTTCCCAAGA |
| 13 | Probe for Campylobacter showae (C.s.) | | CAATGGGTATTCTTCTTGAT |
| 14 | Probe for Fusobacterium nucleatum subsp. | | TAGTTATACAGTTTCCAACG |
| | vincenti (F.n.) | | |
| 15 | Probe for Fusobacterium nucleatum subsp. | | CTAGTTATACAGTTTCCAAC |
| | vincenti (F.n.) | | |
| 16 | Probe for Fusobacterium nucleatum subsp. | | TCCAGTACTCTAGTTACACA |
| | polymorphum (F.n.) | | |
| 17 | Probe for Fusobacterium nucleatum subsp. | | CCAGTACTCTAGTTACACA |
| | polymorphum (F.n.) | | |
| 18 | Probe for Fusobacterium nucreatum subsp. | | TTTCTTTCTTCCCAACTGAA |
| | animalis (F.n.) | | |
| 19 | Probe for Fusobacterium nucleatum subsp. | | ATTTCTTTCTTCCCAACTGA |
| | animalis (F.n.) | | |
| 20 | Probe for Fusobacterium nucleatum subsp. | | TACATTCCGAAAAACGTCAT |
| | nucleatum (F.n.) | | |
| 21 | Probe for Fusobacterium nucleatum subsp. | | TTACATTCCGAAAAACGTCA |
| | nucleatum (F.n.) | | |
| 22 | Probe for Fusobacterium periodonticum | | TATGCAGTTTCCAACGCAA |
| | (F.p.) | | |
| 23 | Probe for Fusobacterium periodonticum | | CTCTAGTTATGCAGTTTCC |
| | (F.p.) | | |
| 24 | Probe for Parvimonas micra (P.m.) | | AAGTGCTTAATGAGGTTAAG |
| 25 | Probe for Parvimonas micra (P.m.) | | TTTCAAGTGCTTAATGAGGT |
| 26 | Probe for Prevotella intermedia (P.i.) | | GGGTAAATGGAAAAAGGCA |
| 27 | Probe for Prevotella intermedia (P.i.) | | GCAAGGTAGATGTTGAGCA |
| 28 | Probe for Prevotella nigrescens (P.n.) | | CTTTATTCCCACATAAAAGC |
| 29 | Probe for Prevotella nigrescens (P.n.) | | TCCTTATTCATGAGGTACAT |
| 30 | Probe for Streptococcus constellatus | | AAGTACCGTCACTGTGTG |
| | (S.c.) | | |
| 31 | Probe for Streptococcus constellatus | | TTAAGTACCGTCACTGTGT |
| | (S.c.) | | |
| 32 | Probe for Aggregatibacter | | GTCAATTTGGCATGCTATTA |
| | actinomycetemcomitans (A.a.) | | |
| 33 | Probe for Aggregatibacter | | TTTAACGTCAATTTGGCATG |
| | actinomycetemcomitans (A.a.) | | |
| 34 | Probe for Campylobacter concisus (C.c.) | | CCCAAGCAGTTCTATGGT |
| 35 | Probe for Campylobacter concisus (C.c.) | | TCCCAAGCAGTTCTATGG |
| 36 | Probe for Capnocytophaga gingivalis | | TACACGTACACCTTATTCTT |
| | (C.g.) | | |
| 37 | Probe for Capnocytophaga gingivalis | | CATCAATGTACACGTACAC |
| | (C.g.) | | |
| 38 | Probe for Capnocytophaga ochracea (C.o.) | | CATTCAAGACCAACAGTTT |
| 39 | Probe for Capnocytophaga ochracea (C.o.) | | CAACCATTCAAGACCAACA |
| 40 | Probe for Capnocytophaga sputigena | | GCTTAGTTGAGCTAAGCG |
| | (C.s.) | | |
| 41 | Probe for Capnocytophaga sputigena | | TCAAAGGCAGTTGCTTAGT |
| | (C.s.) | | |
| 42 | Probe for Eikenella corrodens (E.c.) | | CTAGCTATCCAGTTCAGAA |
| 43 | Probe for Eikenella corrodens (E.c.) | | CTCTAGCTATCCAGTTCAG |
| 44 | Probe for Streptococcus gordonii (S.g.) | S.s. | CACCCGTTCTTCTCTTACA |
| 45 | Probe for Streptococcus gordonii (S.g.) | S.s. | CACCCGTTCTTCTCTTAC |
| 46 | Probe for Streptococcus intermedius | | CAGTATGAACTTTCCATTCT |
| | (S.i.) | | |
| 47 | Probe for Streptococcus intermedius | | ACAGTATGAACTTTCCATTCT |
| | (S.i.) | | |
| 48 | Probe for Streptococcus mitis (S.m.) | S.m. by | CCCCTCTTGCACTCAAGT |
| | | 2, S.o. | |
| 49 | Probe for Streptococcus mitis (S.m.) | | TCTCCCCTCTTGCACTCA |
| 50 | Probe for Streptococcus mitis by 2 | S.o. | TCCCCTCTTGCACTCAAGT |
| | (S.m.) | | |
| 51 | Probe for Actinomyces odontolyticus | | AAGTCAGCCCGTACCCA |
| | (A.o.) | | |
| 52 | Probe for Actinomyces odontolyticus | | CGCACTCAAGTCAGCCC |
| | (A.o.) | | |
| 53 | Probe for Veillonella parvula (V.p.) | | CTATTCGCAAGAAGGCCTT |
| 54 | Probe for Veillonella parvula (V.p.) | | TATTCGCAAGAAGGCCTT |
| 55 | Probe for Actinomyces naeslundii II | | CACAAGGAGCAGGCCTG |
| | (A.n.) | | |
| 56 | Probe for Actinomyces naeslundii II | | CCACCCACAAGGAGCAG |
| | (A.n.) | | |
| 57 | Probe for Selenomonas noxia (S.n.) | | TTCGCATTAGGCACGTTC |
| 58 | Probe for Selenomonas noxia (S.n.) | | CTATTCGCATTAGGCACGT |
| 59 | Probe for Streptococcus mutans (S.mu.) | | CACACGTTCTTGACTTAC |
| 60 | Prober for total bacteria amount | | CGTATTACCGCGGCTGCTGGCAC |
| | (16S rRNA consensus seq.) | | |
| 61 | Prober for external control Ctrl1 | | CGTGCATTGTCGTGTAGGTTCGACCCTAAT |
| 62 | Prober for external control Ctrl2 | | GCAGCTACGTTCATACCTACGCAAGGCATT |
| 63 | Prober for external control Ctrl3 | | GAGGAGATACCGAATCGGTCGACGACATTT |

### <Preparation of Test Sample>

Next, a thermal cycler was turned on and the program was set as shown in Table 3.

TABLE 3
Ramp max, Liquid amount 20 μl

| 95° C. | 95° C. | 56° C. | 72° C. | 40° C. | 4° C. |
|---|---|---|---|---|---|
| 1 min | 10 sec | 30 sec | 20 sec | 8 min | ∞ (limitless) |

40 Cycles

By using ethanol and Kimwipe/Kimtowel or the like for sterilization, rubber gloves, bench top, pipette man, tube rack, or the like were carefully cleaned and the tube rack was placed on ice. On the tube rack, a sample for evaluation, primer (2 primers), Ex Taq, an externally added control, and nuclease-free water were placed. If necessary, a chemical reagent was dissolved, vortexed, and spun down. An Eppendorf tube for producing premix, an Eppendorf tube for diluting primer, a tube for diluting an externally added control, and an Eppendorf tube for carrying out the reaction (0.2 mL, portion of number of samples) were placed in the tube rack. Each of the primers was diluted to have 10 pmol/μL and added to the Eppendorf tube for diluting primer.

### <Preparation of Premix for PCR>

To an Eppendorf tube, water was added at number of samples×6 μL. The externally added control which has been diluted by 100,000 was added at number of samples×1 μL. 10 pmol/μL primer (reverse) was added at number of samples×1 μL. 50 pmol/μL primer (forward) was added at number of samples×1 μL. 2×Taq was added at number of samples×10 μL. After vortexing and spinning-down, a premix was obtained.

### <Preparation of Reaction Solution>

19 μL of the premix were aliquoted in an Eppendorf tube for reaction (0.2 mL), and to an Eppendorf tube for reaction, the sample shown in the following table was added in an amount of 1 μL. After vortexing and spinning-down, a reaction solution was obtained.

As for the sample, a lyophilized bacterial strain or genomic DNA purchased from ATCC was used. The samples for use are shown in Table 4. DNA was extracted by using Dneasy Blood & Tissue Kit (QIAGEN) after adjusting the genomic DNA to 10 ng/μL or dissolving the bacterial strain in 300 μL. As for the concentration, the respectively required range was measured from 0.01 to 10 pg.

TABLE 4

| Type of bacteria | ATCC No. |
|---|---|
| Prevotella nigrescens Shah and Gharbia | 33563 |
| Capnocytophaga ochracea Leadbetter et al. | 33596 |
| Eikenella corrodens (Eiken) Jackson and Goodman | 23834 |
| Streptococcus mitis Andrewes and Horder emend. | 49456D-5 |
| Judicial Commission | |
| Actinomyces naeslundii Thompson and Lovestedt | 12104D-5 |
| Porphyromonas gingivalis | 33277D |
| Tannerella forsythensis | 43037D-5 |
| Treponema denticola | 35405D-5 |
| Prevotella intermedia | 25611D-5 |
| Haemophilus actinomycetemcomitans | 700685D-5 |
| Fusobacterium periodonticum | 33693 |
| Streptococcus intermedius | 27335 |
| Selenomonas noxia | 43541 |
| Campylobacter concisus | BAA-1457D-5 |
| Campylobacter gracilis | 33236D-5 |
| Fusobacterium nucleatum subsp. Animalis | 51191 |
| Fusobacterium nucleatum subsp. polymorphum | 10953 |
| deposited as Fusobacterium polymorphum | |
| Fusobacterium nucleatum subsp. Vincenti | 49256 |
| Streptococcus gordonii | 35105D-5 |
| Campylobacter rectus; Strain FDC 371 | 33238D-5 |
| Capnocytophaga sputigena | 33612 |

### <PCR>

The reaction solution was set on a thermal cycler. After tightly closing the cover of the thermal cycler, the start button was pushed to initiate the reaction.

### <Preparation for Hybridization>

After turning on a thermal cycler, the program was set [95° C., 5 min (if necessary, allowed to stand for some time at 4° C.), amount of reaction solution: 100 μL, RampSpeed: Max]. To a rather small vessel (with a size capable of storing a 96 well plate), ice was added and pure water was added to fill evenly any spaces between the ice.

### <Preparation of Premix for Hybridization>

A 1.5 mL Eppendorf tube or a 8 mL conical tube was prepared. To the tube, clean water was added at number of samples×1.1×64 μL. To the tube, 1 M Tris-HCl was added at number of samples×1.1×48 μt. To a tube, 1 M NaCl was added at number of samples×1.1×48 To a tube, 0.5% Tween20 was added at number of samples×1.1×20 μL. After thorough vortexing and spinning-down, a premix for hybridization was obtained.

### <Preparation of Solution for Hybridization>

To a sample solution (i.e., 0.2 mL Eppendorf tube added with PCR product), 180 μL of the premix for hybridization was aliquoted. After vortexing and spinning-down, a hybridization solution was obtained.

### <Hybridization Pretreatment>

The hybridization solution was set on a thermal cycler. After tightly closing the cover of the thermal cycler, heating was initiated. When the heating for 5 minutes at 95° C. is completed, the cover was open, and the sample tube inside the cycler was removed together with the rack. After placing in a rather small vessel added with ice, the sample tube was allowed to stand for 2 minutes. After that, the sample tube was removed from ice water and place in a rack on ice.

### <Hybridization>

After confirming that an air incubator is set at 50° C., the entire amount (200 μL) of the hybridization solution was added to the hybridization chamber. Then, it was impregnated in the Genopal hybridization chamber which has been prepared in the above. In a sealed state after covering the hybridization chamber, it was added to the air incubator and allowed to stand in the same state for 2 hours under light-shielding conditions.

### <Washing>

After 2 hours, the hybridization chamber was removed from the air incubator and the cover was removed. A conical tube added with 0.24 M TNT buffer was removed from a water incubator, and to remove water drops attached on the cover, it was rotated while the conical tube was covered with a cover. After that, the cover was removed. From the hybridization chamber, the chip was removed using a forceps, and the bottom surface was contacted with Kimwipe or the like to absorb and remove excess hybridization solution. The chip was added to the conical tube, covered again, and impregnated in the water incubator. It was heated for 20 minutes in the same state. After 20 minutes, the chip was transferred to another conical tube added with the same buffer according to the same method as above. Heating was carried again for 20 minutes. After 20 minutes, this time the chip was transferred to a conical tube added with 0.24 M TN buffer according to the same method as above, and heating was carried out again for 10 minutes. After 10 minutes, the chip was transferred to a 8 mL conical tube added with the same buffer according to the same method as above, and then rotated and stirred for 10 minutes or longer using a rotating flat form.

### <Operation for Detection>

The chip was removed from the conical tube and set on a detection case for Genopal Reader (manufactured by Mitsubishi Rayon Co., Ltd.).

By using Genopal Reader (manufactured by Mitsubishi Rayon Co., Ltd.), photographic images of the DNA micro array were taken according to the manuals for handling the apparatus at light exposure time conditions of 40 seconds, 4 seconds, 1 second, or 0.1 second. Then, the numerical value data converted based on detected fluorescent signal was used for analysis.

### <Analysis>

By using a commercially available software for calculating tables, in addition to the name of a sample, information regarding the sample, chip, or experimental conditions, and numerical values of the signal intensity were supplied to a PC.

The average value of several background spots excluding outlier (i.e., spots not mounted with probe) was used as a background value. Furthermore, the standard deviation value at that time was used as a minimum signal value.

From the probe spot signal value, the background value was subtracted to obtain a net signal value.

When a net signal value is lower than the minimum signal value, it was replaced with the minimum signal value.

The results are shown in Table 5 (Table 5-1 to 5-5; Table 5 corresponds to Table 5-1 of which left end is connected to Table 5-2 to 5-5 in order). Among the data, those in small enclosing boxes indicate the signal derived from bacterial species-specific hybridization. Although cross hybridization is partially shown here and there, a strong signal was detected from the probe for detecting the corresponding bacterial species and the total mount indicator probe in most of the samples.

Next, from the signal intensity of probe for each bacterial species, hybridization efficiency coefficient of each probe was obtained. For the probe which forms hybridization with several bacteria, the average value was taken as the hybridization efficiency coefficient, and the results are shown in Table 6. Meanwhile, absence of description indicates that, as there were no isolated bacteria or nucleic acids, the hybridization efficiency coefficient cannot be obtained.

TABLE 6

| | | Hybridization | |
|---|---|---|---|
| | | coefficient | |
| | | [SLOPE | |
| | | (pg/SI)] | RSQ |
| Probe | Porphyromonas gingivalis | 1 × 10⁻⁴ | 0.98 |
| name | Tannerella forsythensis | 1 × 10⁻⁴ | 1.00 |
| | Treponema denticola | 8 × 10⁻⁵ | 1.00 |
| | Campylobacter rectus | 6 × 10⁻⁶ | 0.99 |
| | Genus Fusobacterium | 6 × 10⁻⁵ | 0.94 |
| | Prevotella intermedia | 5 × 10⁻⁴ | 0.81 |
| | Prevotella nigrescens | 2 × 10⁻⁴ | 1.00 |
| | Shah and Gharbia | | |
| | Haemophilus actinomycetemcomitans | 9 × 10⁻⁶ | 0.88 |
| | Campylobacter concisus | 1 × 10⁻³ | 0.75 |
| | Capnocytophaga gingivalis, | 4 × 10⁻⁶ | 0.76 |
| | Capnocytophaga sputigena | | |
| | Capnocytophaga ochracea | 5 × 10⁻³ | 0.38 |
| | Leadbetter et al. | | |
| | Eikenella corrodens | 4 × 10⁻³ | 0.56 |
| | (Eiken) Jackson and Goodman | | |
| | Streptococcus gordonii, | 5 × 10⁻⁶ | 0.92 |
| | Streptococcus mitis | | |
| | Actinomyces naeslundii | 2 × 10⁻² | 0.94 |
| | Thompson and Lovestedt | | |
| | Streptococcus mutans | 5 × 10⁻⁶ | 0.91 |
| | Total bacteria amount | 1 × 10⁻⁴ | 0.82 |

### EXAMPLE 2

### <Preparation of Externally Added Control>

Double strand DNA shown in SEQ ID NOs: 76 to 90, which can be amplified by a forward primer and a reverse primer and each can be separately hybridized using the probes of SEQ ID NOs: 61 to 75, was synthesized. The synthesized sequences are shown in Table 7.

TABLE 7

| SEQ | | | |
|---|---|---|---|
| ID | | [copy/ | |
| NO | Name | ξI] | Sequence (5′→3′) |
| 76 | External | 10 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl1 | | CTCGTCTTATCCCGAACTTGGCATCTGCTGATACGTCAGGTTGAACGCGTACATTTACCTGTCATGC |
| | | | GTGAATGGTAAGGGTCGTATTAGGGTCGAACCTACACGACAATGCACGTCGAAGCGGTTGCTAATG |
| | | | CACATTGGCTAAGGCCCACGGAACACGAATCACGTGAGATCACTTACTATTCGACGGAACTACTATA |
| 77 | External | 20 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl2 | | CTCGTCTTATCCCGAACTTGGCATCTGCTGATACGTCAGGTTGAACGCGTACATTTACCTGTCATGC |
| | | | GTGGTAGCCGATTGAACTAATGCCTTGCGTAGGTATGAACGTAGCTGCTAGTCGAGGCCTTGTATG |
| | | | CACATTGGCTAAGGCCCACGGAACACGAATCACGTGAGATCACTTACTATTCGACGGAACTACTATA |
| 78 | External | 40 | GTGAGAAGCCTACACAAACGTAACGTGAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl3 | | CTCGTCTTATCCCGAACTTGGCATCTGCTGATACGTCAGGTTGAACGCGTACATTTACCTGTCATGC |
| | | | GTGTCCTCGATACATACGAAATGTCGTCGACCGATTCGGTATCTCCTCTGGCACCGAAGACAAATG |
| | | | CACATTGGCTAAGGCCCACGGAACACGAATCACGTGAGATCACTTACTATTCGACGGAACTACTATA |
| 79 | External | 80 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl4 | | CTCGTCTTATCCCGAACTTGGCATCTGCTGATACGTCAGGTTGAACGCGTACATTTACCTGTCATGC |
| | | | GTGTGCTACGCTTTACGCTTGCCAATCGTTCAGGACCTTCACGCAACACTTGATCGAACCGAAATG |
| | | | CACATTGGCTAAGGCCCACGGAACACGAATCACGTGAGATCACTTACTATTCGACGGAACTACTATA |
| 80 | External | 160 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl5 | | CTCGTCTTATCCCGAACTTGGCATCTGCTGATACGTCAGGTTGAACGCGTACATTTACCTGTCATGC |
| | | | GTGACAACAGACGGTACGTCCATTAGTGCAACGTTTGCTCAGTAGGGGGTCTAAGCGTGACTTATG |
| | | | CACATTGGCTAAGGCCCACGGAACACGAATCACGTGAGATCACTTACTATTCGACGGAACTACTATA |
| 81 | External | 320 | GTGAGAAGCCTAGACAAACGTAACGTCAGGGCTAAGACAAAGGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl6 | | CTCGTCTTATCCCGAACTTGGCATCTGCTGATACGTCAGGTTGAACGCGTACATTTACCTGTCATGC |
| | | | GTGGGAGCAGTTTTCTTCTCGTACGTGACTATGCACTCGGTCGTTGTTGGAATACCGGTCGTAATG |
| | | | CACATTGGCTAAGGCCCACGGAACACGAATCACGTGAGATCACTTACTATTCGACGGAACTACTATA |
| 82 | External | 640 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl7 | | CTCGTCTTATCCCGAACTTGGCATCTGCTGATACGTCAGGTTGAACGCGTACATTTACCTGTCATGC |
| | | | GTGAACGCAATACGGTGGGACTTTTCTCGCCAATACCTTAGGGCTCCTGTGTACCTAAGCGAAATG |
| | | | CACATTGGCTAAGGCCCACGGAACACGAATCACGTGAGATCACTTACTATTCGACGGAACTACTATA |
| 83 | External | 1280 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTGACGAAGGCAATTCCCTGAAAGC |
| | Ctrl8 | | CTCGTCTTATCCCGAACTTGGCATCTGCTGATACGTCAGGTTGAACGCGTACATTTACCTGTCATGC |
| | | | GTGGGATGACGGATAAGTTGCAACCTCGGAAGATATGCGGATACTCAGACGTGATATGCGCAGATG |
| | | | CACATTGGCTAAGGCCCACGGAACACGAATCACGTGAGATCACTTACTATTCGACGGAACTACTATA |
| 84 | External | 2560 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl9 | | CTCGTCTTATCCCGAACTTGGCATCTGCTGATACGTCAGGTTGAACGCGTACATTTACCTGTCATGC |
| | | | GTGGGTGTACATCGGATGACAGCGTTATGGTCCTTCGGTCAGCTAAGTAAGTCCGTTTTCCACATG |
| | | | CACATTGGCTAAGGCCCACGGAACACGAATCACGTGAGATCACTTACTATTCGACGGAACTACTATA |
| 85 | External | 5120 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl10 | | CTCGTCTTATCCCGAACTTGGCATCTGCTGATACGTCAGGTTGAACGCGTACATTTACCTGTCATGC |
| | | | GTGTTCAGTCAACCGGAGAAGTCAACGGTTGACTACGGATCCCTTCCATGTAGAGCTCTCACAATG |
| | | | CACATTGGCTAAGGCCCACGGAACACGAATCACGTGAGATCACTTACTATTCGACGGAACTACTATA |
| 86 | External | 10240 | GTGAGAAGGCTACACAAACGTAACGTCAGGGCTAAGACAAACGCTAACGGTACACCCTAGATGGGA |
| | control | | GCTTGTAGCTAGATCGCTAAGTCCTACCGAGATGTAGGCATACTCACGAAGGCAATTCCCTGAAAGC |
| | Ctrl11 | | CTCGTCTTATCCCGAACTTGGCATCTGCTGATACGTCAGGTTGAACGCGTACATTTACCTGTCATGC |
| | | | GTGGCGATTGTCGCGTTAAACATTCTGTAGGCGTCGTATGTCGATCCGGGACTTCGCTTCATAATG |
| | | | CACATTGGCTAAGGCCCACGGAACACGAATCACGTGAGATCACTTACTATTCGACGGAACTACTATA |
| 87 | External | 20480 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl12 | | CTCGTCTTATCCCGAACTTGGCATCTGCTGATAGGTCAGGTTGAAGGCGTACATTTACCTGTCATGC |
| | | | GTGTCGTGTGTCGTGAGAGGAGCACTCATAGTCTCGCGTAGACGTTTATGACGAGATATCACGATG |
| | | | CACATTGGCTAAGGCCCACGGAACACGAATCACGTGAGATCACTTACTATTCGACGGAACTACTATA |
| 88 | External | 40960 | GTGAGAAGCCTACACAAACGTAACGTCAGGGCTAAGACAAACGCTAACGGTACACCCTAGATGGGA |
| | control | | GCTTGTAGCTAGATCGCTAAGTCCTACCGACATGTAGGCATACTCACGAAGGCAATTCCCTGAAAGC |
| | Ctrl13 | | CTCGTCTTATCCCGAACTTGGCATCTGCTGATACGTCAGGTTGAACGCGTACATTTACCTGTCATGC |
| | | | GTGGTAGCGTGTAACGCACTAATGTGGTACGTCGGATCGATCCATACGCAACTTTGTACCGAGATG |
| | | | CACATTGGCTAAGGCCCACGGAACACGAATCACGTGAGATCACTTACTATTCGACGGAACTACTATA |

### <From PCR to Data Obtainment>

By using 500 μL of saliva from 1 person as a test subject, who has been recruited from the applicant company, instead of bacterial strain or DNA solution, DNA was extracted with a use of Dneasy Blood & Tissue Kit (QIAGEN). The concentration was 14.8 ng/μL. The actual sample was 1 μL obtained by dilution of 3000 times, and 1 μL of the externally added control which has been diluted by 100,000 times was added thereto. Other than that, the PCR was carried out in the same order and the same method as Example 1 except that the amount of water in the reaction solution was reduced by 1 μL per sample. After subsequently performing the hybridization using a DNA micro array, a fluorescent signal data was obtained for each probe. The results obtained with the probes from which a signal higher than the detection limit has been obtained are summarized in Table 8.

TABLE 8

| | | | Copy | Copy |
|---|---|---|---|---|
| | | | number, | number, |
| | | | 3000 × | before |
| | | SI Median | diluted | dilution |
| | | [nM/m²] | solution | [copy] |
| Probe | Porphyromonas gingivalis | 0 | 0 | 0 |
| name | Tannerella forsythensis | 11 | 1 | 1794 |
| | Treponema denticola | 121 | 4 | 13101 |
| | Campylobacter rectus | 0 | 0 | 0 |
| | Genus Fusobacterium | 2709 | 65 | 195095 |
| | Prevotella intermedia | 0 | 0 | 0 |
| | Prevotella nigrescens | 54 | 6 | 17112 |
| | Shah and Gharbia | | | |
| | Haemophilus | 0 | 0 | 0 |
| | actinomycetemcomitans | | | |
| | Campylobacter concisus | 0 | 0 | 0 |
| | Capnocytophaga gingivalis, | 0 | 0 | 1 |
| | Capnocytophaga sputigena | | | |
| | Capnocytophaga ochracea | 0 | 0 | 0 |
| | Leadbetter et al. | | | |
| | Eikenella corrodens | 0 | 0 | 0 |
| | (Eiken) Jackson and Goodman | | | |
| | Streptococcus gordonii, | 258 | 1 | 1835 |
| | Streptococcus mitis | | | |
| | Actinomyces naeslundii | 43 | 285 | 854334 |
| | Thompson and Lovestedt | | | |
| | Streptococcus mutans | 409 | 1 | 2902 |
| | Total bacteria amount | 25292 | 1368 | 4105165 |

The results of Table 8 indicate copy number for each probe which has been calculated by using the hybridization efficiency coefficient obtained in Example 1.

In Table 8, the numerical value described in the column of “copy number” means the number of actual bacteria.

The slope value of the calibration curve, which has been established within the quantification range of the absolute amount indicator probe (i.e., data excluding the data from the probes lower than detection limit and the data from the probes having signal intensity as a limit point), was calculated. The results are shown in Table 9.

TABLE 9

| Probe | External control as | Added copy number | SI Median |
|---|---|---|---|
| (SEQ ID NO.) | detection subject | [copy/μl] | [nW/m²] |
| 58 | External control Ctrl1 | 10 | 154 |
| 60 | External control Ctrl3 | 40 | 308 |
| 64 | External control Ctrl7 | 640 | 956 |
| 66 | External control Ctrl9 | 2560 | 3122 |
| 68 | External control Ctrl11 | 10240 | 13759 |
| 72 | External control Ctrl15 | 163840 | 53059 |

From the above, it was shown that the absolute quantification of bacteria as a detection subject, which are contained in a saliva sample of a person as a test subject, can be achieved.

### INDUSTRIAL APPLICABILITY

According to the present invention, each of plural bacteria contained in an environment can be quantified in batch mode, and also an increase or a decrease in the overall amount can be evaluated. Thus, the device of the present invention can be used for evaluation of gut, skin, or oral health state, evaluation of an environment including soil, seawater, and stream, and evaluation of active sludge or the like.

### [Sequence Listing Free Text]

SEQ ID NOs: 1 to 92: Synthetic nucleic acids

1. A device mounted with the following probe (a) and at least one of probe (b) and (c):
(a) a probe consisting of a nucleic acid hybridizing to 16SrRNA specific to each of 1 kind or 2 or more kinds of bacteria to be a subject of detection,
(b) a total amount indicator probe,
(c) one or more kinds of an absolute amount indicator probe.

2. The device according to claim 1, wherein the probe (b) contains a base sequence which is commonly possessed by the 1 kind or 2 or more kinds of bacteria.

3. The device according to claim 1, wherein the probe (c) contains a base sequence which captures a mixture of at least one kind of a predetermined externally added control, and wherein the mixture contains the at least one kind of a predetermined externally added control and at least one kind of a predetermined externally added control at a concentration ratio of 2ⁿ, wherein n is an arbitrary integer, relative to each other.

4. The device according to claim 1, wherein the bacteria to be a subject of detection are enterobacteria, resident bacteria of skin, or oral bacteria.

5. The device according to claim 4, wherein the enterobacteria are at least one of genus Lactobacillus, genus Streptococcus, genus Veionella, genus Bacteroides, genus Eubacterium, genus Bifidobacterium, and genus Clostridium.

6. The device according to claim 4, wherein the resident skin bacteria are at least one of genus Propionibacterium and genus Staphylococcus.

7. The device according to claim 4, wherein the oral bacteria are at least one of genus Porphyromonas, genus Tannerella, genus Treponema, genus Campylobacter, genus Fusobacterium, genus Parvimonas, genus Streptococcus, genus Aggregatibacter, genus Capnocytophaga, genus Eikenella, genus Actinomyces, genus Veillonella, genus Selenomonas, genus Lactobacillus, genus Pseudomonas, genus Haemophilus, genus Klebsiella, genus Serratia, genus Moraxella, and genus Candida.

8. The device according to claim 7, wherein the oral bacteria are at least one of Porphyromonas gingivalis, Tannerella forsythia, Treponema denticola, Prevotella intermedia, Aggregatibacter actinomycetemcomitans, Fusobacterium nucleatum subsp. vincentii, Fusobacterium nucleatum subsp. polymorphum, Fusobacterium nucleatum subsp. animalis, Fusobacterium nucleatum subsp. nucleatum, Streptococcus mutans, Streptococcus salivarius, Streptococcus sanguis, Streptococcus miris, Actinomyces viscosus, Lactobacillus gasseri, Lactobacillus phage, Lactobacillus casei, Lactobacillus delbrueckii subsp. bulgaricus, Streptococcus aureus, Pseudomonas aeruginosa, Streptococcus pyogenes, Streptococcus pneumoniae, Haemophilus influenzae, Klebsiella pneumoniae, Serratia marcescens, Serratia macescens, Moraxella catarrhalis, Candida albicans, Campylobacter gracilis, Campylobacter rectus, Campylobacter showae, Fusobacterium periodonticum, Parvimonas micra, Prevotella nigrescens, Streptococcus constellatus, Campylobacter concisus, Capnocytophaga ngivalis, Capnocytophaga ochracea, Capnocytophaga sputigena, Eikenella corrodens, Streptococcus gordonii, Streptococcus intermedius, Streptococcus mitis by 2, Actinomyces odontolyticus, Veillonella parvula, Actinomyces naeslundii II, and Selenomonas noxia.

9. The device according to claim 1, wherein the probe (a) is any one sequence of the following sequences:
(i) at least 1 sequence selected from base sequences represented by SEQ ID NOs: 3 to 59,
(ii) a sequence complementary to (i), or
(iii) a sequence substantially the same as the sequence of (i) or (ii).

10. The device according to claim 1, wherein the device is a fiber type micro array.

11. A probe set, comprising any one sequence of the following sequences:
(i) at least 1 sequence selected from base sequences represented by SEQ ID NOs: 3 to 59, and a base sequences represented by SEQ ID NO: 60,
(ii) a sequence complementary to (i),
(iii) a sequence which comprises the sequence (i) or (ii), or
(iv) a sequence as a part of the sequence (i) or (ii).

12. A method for estimating an absolute amount of bacterial species as a subject of detection, comprising:
(1) obtaining, for each of bacteria previously isolated as a subject of detection, a coefficient from a signal intensity ratio of a probe for detecting the bacteria as a subject of detection;
(2) correcting a signal intensity obtained from measuring a test sample by comparing signal intensity from the test sample to a signal intensity of an absolute amount indicator probe; and
(3) calculating copy number of each bacterial species as a subject of detection based on a corrected signal intensity of a test sample in step (2) using the coefficient obtained in step (1).
